(19) United States
(12) Patent Application Publication
(10) Pub. No.: US 2015/0216558 A1
(43) Pub. Date: Aug. 6, 2015

(54) TUNNELLING DEVICE WITH TUBE AND OLIVE

(71) Applicant: B. Braun Melsungen AG, Melsungen (DE)

(72) Inventor: Heinz Wiegel, Alheim (DE)

(21) Appl. No.: 14/426,274

(22) PCT Filed: Aug. 28, 2013
(86) PCT No.: PCT/EP2013/067844

§ 371 Date: Mar. 5, 2015

(30) Foreign Application Priority Data
Sep. 6, 2012 (DE) 10 2012 215 847.2

(51) Int. Cl. A61B 17/34; A61M 25/01; A61M 25/09; A61M 25/00

(52) U.S. Cl. CPC A61B 17/3415; A61M 25/0068; A61M 25/01; A61M 25/0194; A61M 25/09; A61M 2025/0175; A61M 2025/09175

(57) ABSTRACT

An improved medical tunnelling device for introducing and positioning a catheter in a patient with a longitudinally extended hollow tube, which has a distal and proximal open end, is characterized in that the distal end is provided with a rounded, rotationally symmetrical thickening.

The invention relates to a medical tunneling device for introducing a catheter into a patient and positioning the catheter in the patient.

Known tunneling devices comprise an elongate hollow tube with a distal and a proximal open end. US 2002/0052576 A1 describes a tunneling device having a guide needle for penetrating the skin of a patient. After the removal of the guide needle, an introduction catheter remains in part under the skin of the patient. An infusion catheter is advanced through the introduction catheter and into the wound area to be treated. When the catheter has been positioned, the introduction catheter is removed and a medicinal solution, typically an anesthetic, is supplied to the wound area via the infusion catheter.

In another known tunneling device, an outside split catheter is used. For placing a pain catheter, the tunneler comprises a steel wire which may cause lesions in the tissue as it is introduced into a patient. After the tunneler has been placed, the same must be withdrawn from the split catheter before the pain catheter can be advanced through the split catheter subsequently. Here, it is a risk that the split catheter leaves the predetermined position as the tunneler is withdrawn. Another known tunneling device is marketed under the designation “ON-Q® Tunneler”. This tunneling device comprises an introduction tube with a blunt distal end so as to reduce damage to the tissue of a patient during the introduction of the tunneling device. However, also a dull end of an introduction tube can cause lesions in the tissue.

It is an object of the invention to provide an improved medical tunneling device bearing a reduced risk of tissue lesioning.

The tunneling device is defined by the features of claim 1.

According thereto, the distal end of the tube (introduction catheter) is provided with a rounded, rotationally symmetrical thickening whose outer diameter is larger than the outer diameter of the tube. The outer diameter of the tube may, for example, be at least 1.5 times and preferably about twice the outer diameter of the tube. Owing to the rounded, rotationally symmetrical shape, the risk of damage to human tissue is reduced during the introduction of the tunneler. When the tube is advanced into the tissue, the latter is displaced sideward by the thickening in any rotational position and under any introduction angle without being destroyed to a significant degree.

Preferably, the thickening is olive-shaped, with the thickening having a substantially oblong shape when seen in a cross section parallel to the central longitudinal axis of the tube. With such a shape, the tissue is displaced sideward in a particularly gentle manner as the tube is advanced and can return to its initial position without being injured in the process.

The distal opening of the tube is formed in a distal end face of the thickening. Given a flexible tube, a guide wire can be advanced through the tube lumen, the guide wire being provided with a rounded distal end. In the initial position prior to the introduction of the tube into a patient, the rounded end of the guide wire protrudes from the distal opening of the tube such that the rounded end of the guide wire completes the roundness of the distal end of the thickening. Thus, a homogenous, blunt and rounded shape is provided that reduces the risk of lesions during the insertion of the tunneling device.

At the proximal end opposite the distal end of the tube, the medical tunneling device may preferably comprise a handle element surrounding the tube, which may be gripped and held by a user as the tunneling device is introduced and positioned. When the tunneling device is introduced, the guide wire is in an initial position in which the distal rounded end of the guide wire protrudes beyond the distal end of the thickening and the tube. The guide wire stabilizes the flexible tube. By means of suitable lever, rotary or advancing movements, the tube is introduced into the patient and the distal end of the tube is placed at the required position. If necessary, the user may (pre-)bend the flexible tube according to the anatomic requirements. Thereafter, the guide wire is removed and the catheter is advanced through the inner lumen of the tube. Finally, the tunneling device is withdrawn from the patient over the catheter so that the catheter remains in the puncture channel. No splittable catheter is required over the tunneling device.

The following is a detailed description of an embodiment of the invention with reference to the drawings. In the FIGURES:

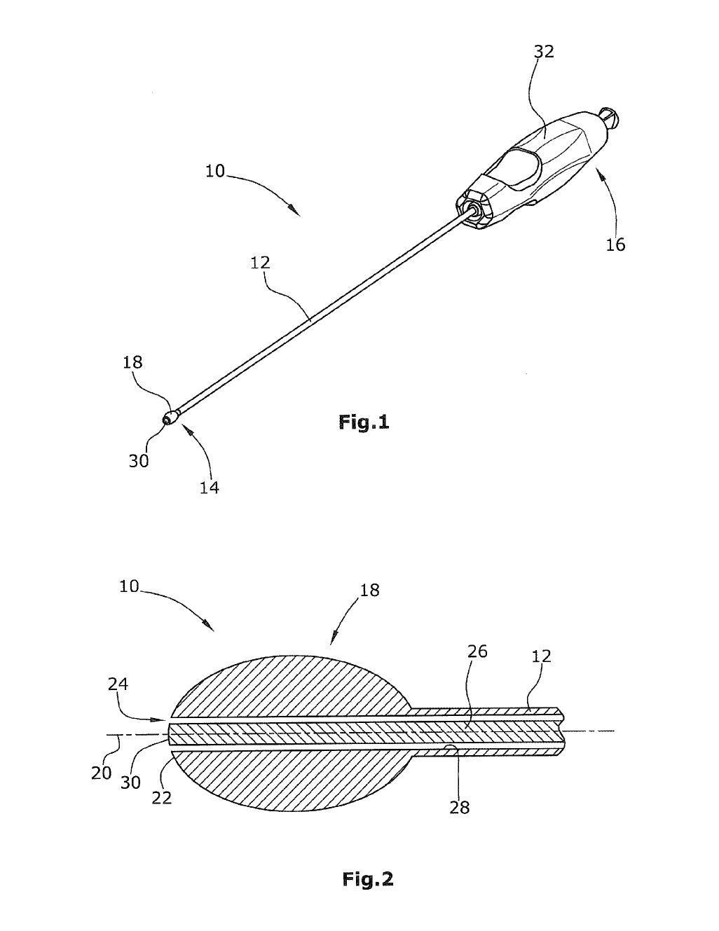

FIG. 1 is a perspective view and

FIG. 2 is a section through the central longitudinal axis of the distal end.

The tunneling device 10 is provided with an elongate hollow tube 12 having a distal end 14 and a proximal end 16 opposite the distal end 14. At the distal end 14, the tube 12 is provided with a rotationally symmetrical, rounded thickening 18. In the section through the central longitudinal axis 20 of the tube 12 illustrated in FIG. 2, it can be seen that the thickening 18 has an approximately oblong cross section and thus an olive-shaped form.

In the distal end face 22 of the thickening 18, the distal opening 24 of the tube 12 is formed, via which the guide wire 26 and the catheter are advanced. The distal opening 24 forms the end 14 of the lumen 28 that extends along the central longitudinal axis 20 and concentrically around the same through the tube 12 and through the thickening 18. The lumen 28 extends entirely through the tube 12 from the distal end 14 thereof through to the proximal end 16 thereof.

The guide wire 26 is contained in the lumen 28. In this regard, the Figures illustrate the initial state of the tunneling device 10 prior to the introduction into a patient. The guide wire 26 is provided with a rounded end 30 that protrudes from the opening 24 slightly beyond the distal end face 22 of the thickening 18. The roundness of the rounded end 30 thus completes the roundness of the thickening 18 to an approximately homogenous olive shape that reduces the risk of injuries during introduction into a patient.

In the region of the proximal end 16 a grip element 32 is formed as a handle 32 on the tube 12. Via the handle 32, the advancing, rotary and lever movements can be performed that are necessary to position the tunneling device 10. Via the rigid, stable guide wire 26, these movements are transferred to the distal end 14 of the tube 12 and to the thickening 18. When the distal tube end 14 has been positioned, the guide wire 26 is removed from the tube 12 and a catheter is then advanced through tube lumen 28.

1. A medical tunneling device for introducing and positioning a catheter in a patient has a elongate hollow tube , which has a distal and a proximal open end, wherein the distal end is provided with a rounded, rotationally symmetrical thickening .

2. The tunneling device of claim 1, wherein the thickening is substantially oblong in shape, seen in a cross section taken in parallel with the central longitudinal axis of the tube, so that the thickening has an olive-like shape.

3. The tunneling device of claim 1, wherein the thickening has the distal opening of the tube provided in its distal end face.

4. The tunneling device of claim 1, wherein the tube is flexible, with a guide wire contained in the tube lumen and removable from the tube, said wire being provided with a rounded distal end.

5. The tunneling device of claim 4, wherein the guide wire is arranged in the tube such that the distal end of the guide wire protrudes from the distal opening of the tube and completes the roundness of the distal end of the thickening.

6. The tunneling device of claim 1, wherein in the region of the proximal end of the tube, a grip element is provided for gripping and guiding the tube, the element surrounding the tube.
